(19) United States
(12) Patent Application Publication
(10) Pub. No.: US 2024/0024169 A1
(43) Pub. Date: Jan. 25, 2024

(54) PANT TYPE ABSORBENT ARTICLE

(71) Applicant: The Procter & Gamble Company, Cincinnati, OH (US)

(72) Inventors: Fancheng WANG, Beijing (CN); Le KANG, Beijing (CN); Koichi MORIMOTO, Cincinnati, OH (US)

(21) Appl. No.: 18/354,697

(22) Filed: Jul. 19, 2023

(30) Foreign Application Priority Data
Jul. 22, 2022 (WO) PCT/CN2022/107306

(51) Int. Cl. A61F 13/49

(52) U.S. Cl. CPC A61F 13/49017; A61F 2013/49092

(57) ABSTRACT

Disposable pull-on garments that provide a pant type absorbent article fit and comfort during wearing as well as an undergarment-like look and feel; wherein the lower front region and the front crotch region have no stretchability, and wherein at least part of the crotch region overlapping the absorbent core has no stretchability

### CROSS-REFERENCE TO RELATED APPLICATIONS

This application claims priority under 35 U.S.C. § 119 to Chinese PCT Patent Application Serial No. PCT/CN2022/107306 filed on Jul. 22, 2022, the entire disclosures of which are hereby incorporated by reference.

### FIELD OF THE INVENTION

The present invention relates to disposable pull-on garments such as menstrual pants, disposable diapers and the like.

### BACKGROUND OF THE INVENTION

Pull-on wearable articles, or pant-type wearable articles, are those which are donned by inserting the wearer's legs into the leg openings and sliding the article up into position about the lower torso. They are intended to fit comfortably and snugly about the wearer. It is further desirable that they solve various kinds of skin troubles at side parts and/or upper leg parts during wearing the article.

Pull-on wearable articles having a main body to cover the crotch region of the wearer and a separate elastic belt defining the waist opening and leg opening are known in the art, such as described in PCT Publication WO 2006/17718A. Such pull-on wearable articles may be referred to as belt-type pants. On the other hand, certain pull-on wearable articles are configured such that the outer cover of the wearable main body completely covers the entirety of the garment-facing surface of the article. Such pull-on wearable articles may be referred to as unibody pants.

It may be desirable in some products to run elastics continuously across the belt in a transversal direction and/or along the entire periphery of a crotch area in order to ensure full elastic fit about the waist and leg. However, running elastics continuously across the belt and/or along the entire periphery of a crotch may create too much tension in force zones and/or elastic sections in the belt and/or crotch area causing uncomfortable tight feel.

Japanese patent 5933193B discloses an underpants type disposable diaper with an exterior sheet comprising a stretchable nonwoven fabric and non-stretchable zone along leg periphery by applying a non-stretchable material. Japanese patent 4290594B discloses a pant-type disposable diaper in which waist elastic members and leg elastic members are not extended to connecting parts on both sides where a front body and a back body are connected, and elasticity by the waist elastic members and the leg elastic members is not imparted in areas of a prescribed width inward from both sides.

There is still an unmet need for providing such a pant type absorbent article fit and comfort during wearing.

There is also a need for a disposable absorbent article to provide an undergarment-like look and feel.

### SUMMARY OF THE INVENTION

Disclosed herein is a disposable absorbent article having a longitudinal direction and a transversal direction perpendicular to the longitudinal direciton, and comprising a front region, a back region, a crotch region between the front region and the back region in the longitudinal direction, and an absorbent core disposed to cover continuously the crotch region and at least part of the front region and back region, wherein the front and back regions are connected each other such that a first and a second seams are formed so that the article comprises leg openings and a waist opening, wherein the front region comprises, in the longitudinal direction, an upper front region, a lower front region, and an intermediate front region between the upper front region and the lower front region, the front region comprising, in the transversal direction, a first side region, a second side region, and a central region between the first side region and the second side region, wherein the crotch region comprise, in the longitudinal direction, a front crotch region connected to the front region, a back crotch region connected to the back region, and an intermediate crotch region between the upper back region and the lower back region, wherein the intermediate front region comprise an area adjacent to each side edge the side edge comprising an elastic material, wherein the lower front region and the front crotch region have no stretchability, and wherein at least part of the crotch region overlapping the absorbent core has no stretchability.

### BRIEF DESCRIPTION OF THE DRAWINGS

While the specification concludes with claims particularly pointing out and distinctly claiming the subject matter which is regarded as forming the present invention, it is believed that the invention will be better understood from the following description which is taken in conjunction with the accompanying drawings in which like designations are used to designate substantially identical elements, and in which:

FIG. 1 is a perspective view of an exemplary disposable pull-on garment of the present invention in a typical in-use configuration.

FIG. 2 is another perspective view of an exemplary disposable pull-on garment of the present invention in a typical in-use configuration.

FIG. 3 is a plan view of an exemplary disposable pull-on garment of the present invention in its flat uncontracted condition showing the wearer-facing surface.

FIG. 4 is a plan view of an exemplary disposable pull-on garment of the present invention in its flat uncontracted condition showing the wearer-facing surface.

FIG. 5 is a schematic cross section view of a first embodiment taken along line 5-5 in FIG. 3 of an exemplary disposable pull-on garment.

FIG. 6 is a schematic cross section view of a second embodiment taken along line 6-6 in FIG. 4 of an exemplary disposable pull-on garment.

FIG. 7 is a schematic cross section view taken along line 7-7 in FIG. 3 of an example of a leg cuff suitable in one embodiment of the invention.

FIG. 8 is a schematic cross section view taken along line 8-8 in FIG. 4 of an example of a leg cuff suitable in one embodiment of the invention.

### DETAILED DESCRIPTION OF THE INVENTION

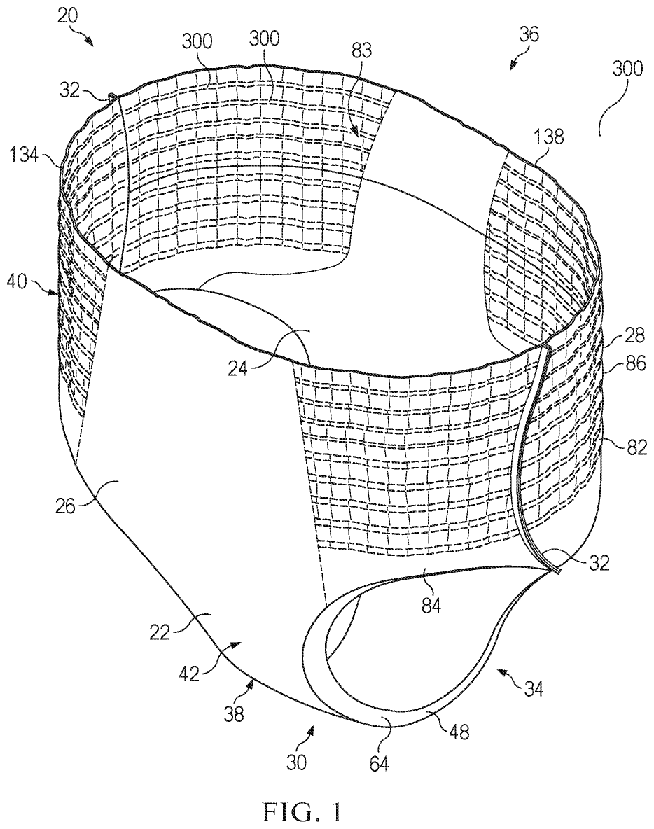

As used herein, the term “pull-on garment” refers to articles of wear which have a defined waist opening and a pair of leg openings and which are pulled onto the body of the wearer by inserting the legs into the leg openings and pulling the article up over the waist. The term “disposable” is used herein to describe garments which are not intended to be laundered or otherwise restored or reused as a garment (i.e., they are intended to be discarded after a single use and, preferably, to be recycled, composted or otherwise disposed of in an environmentally compatible manner). The pull-on garment is also preferably “absorbent” to absorb and contain the various exudates discharged from the body. A preferred embodiment of the absorbent article is the disposable absorbent pull-on garment, shown in FIG. 1.

As used herein, the term “absorbent article” refers to pull-on garments generally worn by infants and other incontinent individuals to absorb and contain urine, feces and/or menses. It should be understood, however, that the term absorbent article is also applicable to other garments such as training pants, incontinent briefs, feminine hygiene garments or panties, and the like.

As used herein, the term “joined” encompasses configurations whereby an element is directly secured to another element by affixing the element directly to the other element, and configurations whereby an element is indirectly secured to another element by affixing the element to intermediate member(s) which in turn are affixed to the other element.

As used herein, a material or region having “no stretchability”, or a material or region is “no stretchable” refers to a material or region that, upon application of a biasing force, can stretch to an elongated length of no more than about 130% of its relaxed, original length (i.e. can stretch no more than 30 percent its original length), without rupture or breakage, and upon release of the applied force, shows little recovery, less than about 20% of its elongation without complete rupture or breakage as measured by EDANA method 20.2-89. In the event such an elongatable material recovers at least 40% of its elongation upon release of the applied force, the elongate-able material will be considered to be “elastic” or “elastomeric.” For example, an elastic material that has an initial length of 100 mm can extend at least to 150 mm, and upon removal of the force retracts to a length of at least 130 mm (i.e., exhibiting a 40% recovery). In the event the material recovers less than 40% of its elongation upon release of the applied force, the elongatable material will be considered to be “substantially non-elastic” or “substantially non-elastomeric”. For example, an elongatable material that has an initial length of 100 mm can extend at least to 150 mm, and upon removal of the force retracts to a length of at least 145 mm (i.e., exhibiting a 10% recovery).

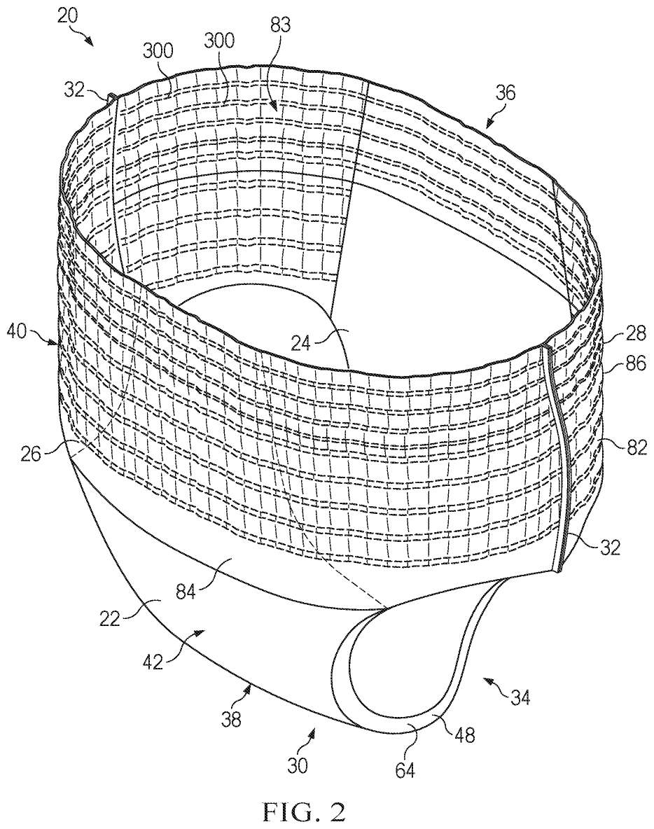

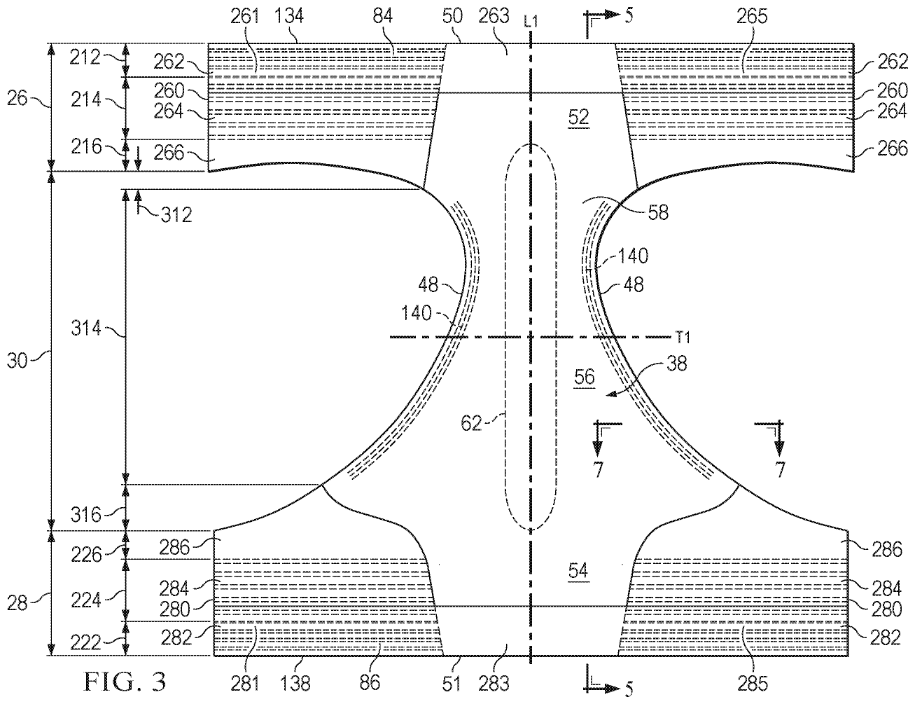

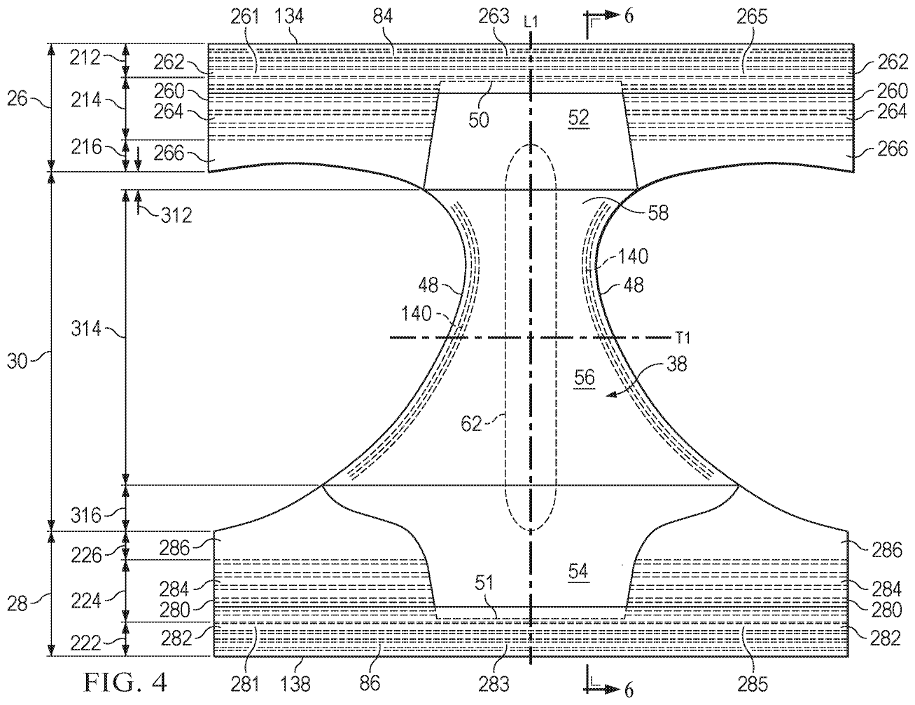

FIG. 1 is a perspective view of a unibody type disposable absorbent article 20 disclosed herein. FIG. 2 is a perspective view of a belt type disposable absorbent article 20 disclosed herein. Referring to FIGS. 3 and 4 as well, absorbent article 20 comprises an outer surface 22 which is a garment-facing surface, an inner surface 24 opposed to the outer surface 22, a front region 26, a back region 28 and a crotch region 30. The absorbent article 20 also comprises seams 32 which join the front region 26 and the back region 28 in their transverse edges to form two leg openings 34 and a waist opening 36. The absorbent article 20 has a longitudinal centerline L1 and a transverse centerline Ti.

As shown in FIGS. 1 and 2, the absorbent article 20 comprises an absorbent main body 38 to cover the crotch region of the wearer and a belt 40 extending transversely about the waist opening 36. The absorbent article 20 may also comprise an outer cover layer 42 to cover the main body 38. The belt 40 defines the waist opening 36. The belt 40, the main body 38 and/or the outer cover layer 42 jointly define the leg opening 34. FIGS. 3 and 4 as well, the absorbent article may comprise leg elastics 140.

The main body 38 is disposed in the crotch region 30, and the front region 26 and back region 28 at least in part in a longitudinal direction so that the front, crotch and back regions are continuous. The front and back regions 26 and 28 may overlap at least a portion of the main body 38. The main body 38 comprises a left and a right longitudinally extending side edges 48 (hereinafter may be referred to as “longitudinal side edge”) and a front and a back transversely extending end edges 50, 51, respectively (hereinafter may be referred to as “front transverse end edge” and “back transverse end edge”, respectively). The main body 38 may be in a general rectangular shape, or be shaped. In one embodiment, the main body 38 is shaped in such a way that side edges of the main body 38 are curved as shown in FIGS. 3 and 4.

The main body 38 also has a front waist panel 52 positioned in the front region 26 of the absorbent article 20, a back waist panel 54 positioned in the back region 28, and a crotch panel 56 between the front and back waist panels 52, 54 in the crotch region 30.

The front transverse end edge 50 of the main body 38 may extend to the front waist edge 134 as shown in FIG. 3, or may extend to the front region 26 and be spaced away from the front waist edge 134 as shown in FIG. 4. The back transverse end edge 51 of the main body 38 may extend to the back waist edge 138 as shown in FIG. 3, or may extend to the back region 28 and be spaced away from the back waist edge 138 as shown in FIG. 4.

The main body 38 comprises a topsheet 58, a backsheet 60 and an absorbent core 62 disposed between the topsheet 58 and the backsheet 60 for absorbing and containing body exudates.

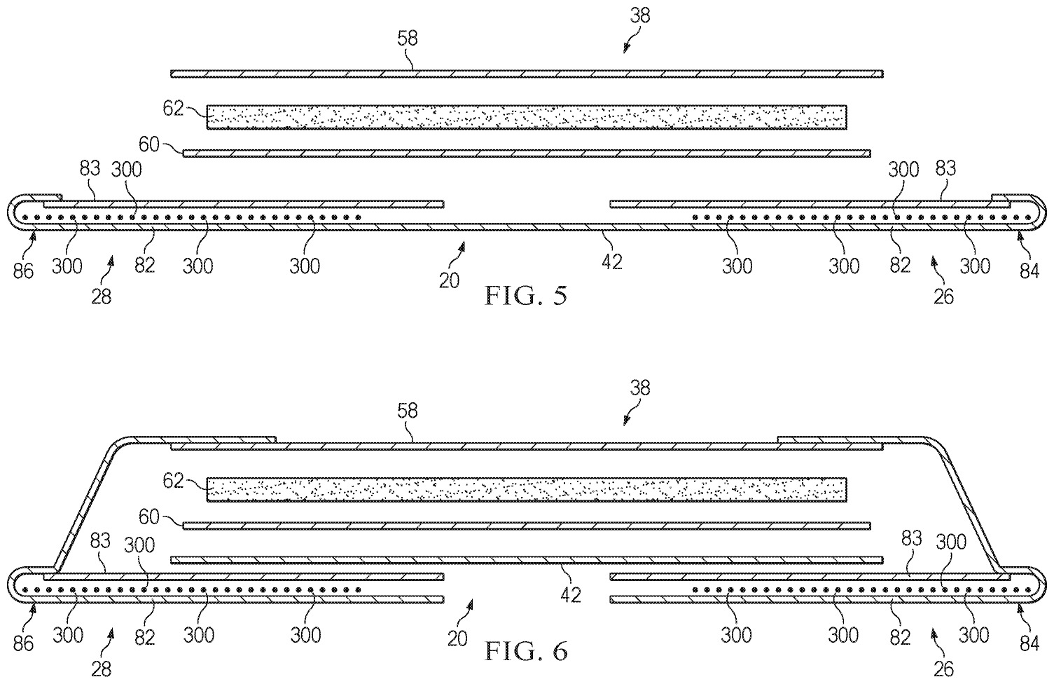

As shown in FIG. 5 showing an exemplary unibody type absorbent article, the absorbent articles 20 may comprise front and back belts 84, 86 disposed in the front and back regions 26, 28 respectively and intended to encircle at least a portion of the waist of the wearer, the front and back belts 84, 86 being connected by the main body 38 forming the crotch region 30 of the article 20. The front and back belts 84, 86 may be formed from a first belt layer 82 extending from a front waist edge 134 in a front region 26 through the crotch region 30 to a longitudinally opposing back waist edge 138 in a back region 28 and forming at least a portion of the outer surface of the absorbent article 20. The front and back belts 84, 86 also may comprise a second belt layer forming a portion of the inner surface 24 of the absorbent article.

The first belt layer 82 may form a portion of the outer surface 22. In the unibody type product, the first belt layer 82 may extend from a front waist edge 134 in a front region 26 through the crotch region 30 to a longitudinally opposing back waist edge 138 in a back region 28 and form a portion or the whole of the outer surface 22 of the absorbent article 20. In one embodiment, referring to FIGS. 3 and 5, the outer cover layer 42 may extend from a front waist edge 134 in a front region 26 through a crotch region 30 to a longitudinally opposing back waist edge 138 in a back region 28 and forming a portion of or the whole of the garment-facing surface 22 of the absorbent article 20. In such a case, the outer cover layer 42 forms the first layer 82.

The second belt layer may overlap at least a portion of the main body and form the outer surface of the first belt layer or alternatively the inner surface of the first belt layer. Alternatively, the inner surface and outer surface of each of the front and back belts is formed from a single web of material. As shown in FIG. 5, the first belt layer 82 may form a portion of the garment facing surface, may fold over and be connected to the second belt layer 83, such that the first belt layer also forms a portion of the wearer facing surface.

As shown in FIG. 6 showing an exemplary belt type absorbent article, the absorbent articles may comprise front and back belts 84, 86 intended to encircle at least a portion of the waist of the wearer, the front and back belts 84, 86 being connected by a main body 38 forming the crotch region 30 of the absorbent article 20. The front and back belts 84 and 86 may be formed from a first belt layer forming a portion of the outer surface 22 of the absorbent article, the first belt layer 82 (also referred to as an outer belt layer) may be formed of two longitudinally spaced webs of material. The front and back belts 84 and 86 may also comprise a second belt layer (also referred to as an inner belt layer) 83 forming a portion of the inner surface 24 of the absorbent article 20. The first and second belt layers 82, 83 may be formed of substantially the same material or may comprise different materials. The first and second belt layers 82, 83 may be formed from nonwovens, films, foams, elastic nonwoven, or combinations thereof. In a belt type absorbent article, the first belt layer may comprise a material different from the outer cover layer.

The front and back belts 84, 86 may also comprise an elastomeric material disposed between the first and second belt layers 82, 83. The elastomeric material may comprise one or more elastic strands, elastomeric films (including apertured films), elastomeric ribbons, elastomeric nonwovens, elastomeric filaments, elastomeric adhesives, elastomeric foams, scrims or combinations thereof. A portion of the elastomeric material may be directly combined with the optional outer cover layer.

In one embodiment, the elastomeric material comprises a plurality of elastics.

Each of the elastics in the front and back belts, as well as the crotch region, may have the same dtex, Dt, (e.g., 800) and the same pre-strain, PS, (e.g., 120%). Another suitable embodiment may have each of the elastic materials of the front and back belts at 470 dtex at about 120 to 150% strain, with lower dtex (smaller diameter of strands) and/or lower pre-strain (less pre-strain) in an area in the crotch region. The front and back belt 84, 86 may comprise any known materials. Suitable material for the front and back belt 84, 86 can be manufactured from a wide range of materials such as plastic films; apertured plastic films; woven or nonwoven webs of natural materials (e.g., wood or cotton fibers); synthetic fibers (e.g., polyolefins, polyamides, polyester, polyethylene, or polypropylene fibers), or a combination of natural and/or synthetic fibers; or coated woven or nonwoven webs. The belt may comprise a nonwoven web of synthetic fibers. The belt may comprise an inner hydrophobic, non-stretchable nonwoven material and an outer hydrophobic, non-stretchable nonwoven material.

The front and back belts 84, 86 may overlap at least a portion of the main body and one or both of the belt portions may be disposed on the outer surface of the main body or alternatively on the inner surface of the main body. A portion of the second belt layer and/or a portion of the first belt layer may be directly attached to the outer cover layer. Alternatively, at least a portion of the inner surface and outer surface of each of the belt portions may be formed from a single web of material. As shown in FIG. 6, the first belt layer 82 may form a portion of the garment facing surface, may fold over and be connected to the second belt layer 83, and may also be connected to a portion of the topsheet 58, such that the first belt layer 82 also forms a portion of the wearer facing surface.

The front region 26 comprises a first side panel and a second side panel and a center panel sandwiched the first and the second side panels in the transversal direction. The back region 28 comprises a first side panel and a second side panel and a center panel sandwiched the first and the second side panels in the transversal direction.

The front and back regions 26, 28 may overlap at least a portion of the main body 38 and one or both of the regions may be disposed on the outer surface of the main body or alternatively on the inner surface of the main body. The center panel of the front region 26 may be at least partially joined to a front waist panel 52 of the main body 38. The center panel of the back region 28 may be at least partially joined to a back waist panel 54 of the main body 38. In the front region 26 and the back region 28, the first and second side panels do not overlap the main body 38.

The front region and back region 26 and 28 may be formed from a first belt layer 82 forming a portion of the outer surface 22 of the absorbent article.

The front and back regions 26 and 28 may also comprise a second belt layer (also referred to as an inner belt layer) forming a portion of the inner surface 24 of the absorbent article 20.

Each of the front region and back region 26 and 28 comprises at least one material continuous along the transversal direction of the absorbent article. In one embodiment, the first belt layer in the front region 26 and/or the back region 28 is continuous along the transversal direction of the absorbent article.

The main body 38 of the absorbent article comprises backsheet 60, topsheet 58, and an absorbent core 62 disposed between the topsheet 58 and the backsheet 60. The backsheet may be formed of a nonwoven material, woven material, films or laminates comprising a combination of one or more of these materials. In one embodiment the backsheet is a film and nonwoven laminate wherein the nonwoven of the laminate is the outer cover layer.

The absorbent article may comprise curved leg elastics that run parallel to, and a few millimeters inboard of, at least a portion of the leg cut outs as shown in FIGS. 3 and 4. The leg elastic material 140 is disposed so as to extend generally longitudinally along the longitudinal side edge 48 of the main body 38. The leg elastic material 140 may be disposed at least in the crotch region 30 of the absorbent article 20 or may be disposed along the entirety of the longitudinal side edge 48.

The main body 38 may additionally comprise elasticized barrier leg cuffs 64 disposed at or adjacent the longitudinal side edges 48 of the main body 38. The main body 38 may additionally comprise a barrier leg cuff 64 disposed along the longitudinal side edge 48 of the main body 38.

The barrier leg cuff 64 provides improved containment of liquids and other body exudates in the crotch region 30.

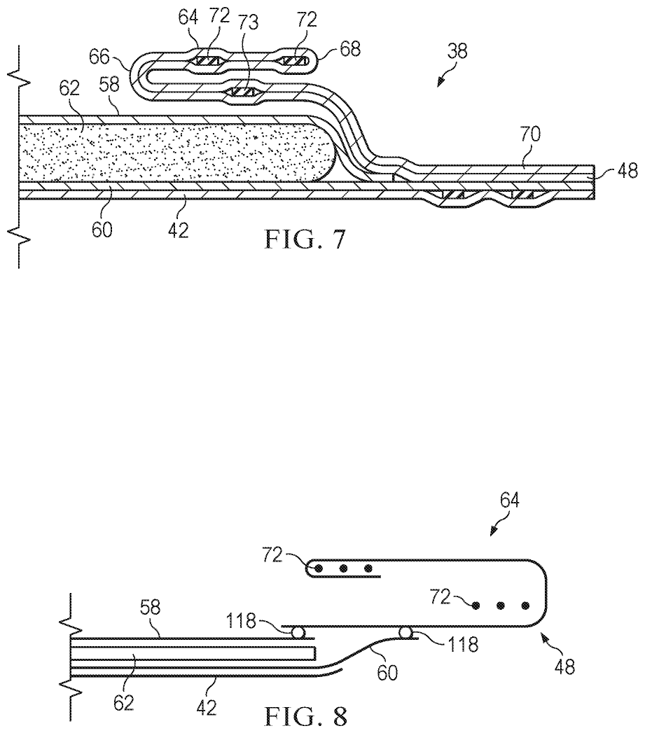

The barrier leg cuff 64 shown in FIG. 7 comprises a single layer of material which may be folded to form a barrier leg cuff having two layers. The barrier leg cuff 64 extends from the side of the main body at or adjacent the longitudinal side edge 48 toward the longitudinal centerline L1. The barrier leg cuff may be folded back toward the longitudinal side edge 48. The barrier leg cuff 64 may have a first barrier cuff elastic material 72 adjacent to the distal portion 68 and a second barrier cuff elastic material 73 adjacent to the proximal portion 70 of the barrier leg cuff 64. The proximal portion 70 of the barrier leg cuff 64 may be joined to the backsheet 60 adjacent to the longitudinal side edge 48. The transverse end of the barrier leg cuff 64 may be joined to the topsheet 58 at or adjacent the longitudinally opposing ends of the leg cuff by an attachment means which may be any known means such as an adhesive, heat bond, pressure bond or the like as shown in FIG. 7.

As shown in FIG. 8, the cuff 64 may have an elastic material 72. A distal portion of the cuff 64 may be adhered to a distal portion of the backsheet 60, and another portion of the cuff 64 may be adhered to the topsheet via adhesive 118.

As shown in FIG. 7, the backsheet 60, the outer cover layer 42, and both layers of the cuff 64 may co-terminate at the side edge 48. Alternatively, as shown in FIG. 8, the distal end of the cuff 64 may extend beyond the other materials to form at least a portion of the side edge 48 in a manner that exposes at least a portion of the cuff when the article 20 is worn, such that a more finished folded leg edge is achieved.

The liquid pervious topsheet 58 may be positioned adjacent the body-facing surface of the absorbent core 62 and may be joined thereto and/or to the backsheet 60 by any attachment means known in the art. The liquid impervious backsheet 60 is generally that portion of the absorbent article 20 positioned adjacent the garment-facing surface of the absorbent core 62 and prevents the exudates absorbed and contained therein from soiling articles that may contact the absorbent article 20. The absorbent core is positioned between the topsheet 58 and the backsheet 60 and absorbs and retains liquids such as urine and other certain body exudates.

The topsheet 58, the backsheet 60 and the absorbent core 62 may be manufactured known materials. Suitable topsheet materials may include porous foams; reticulated foams; apertured plastic films; or woven or nonwoven webs of natural fibers (e.g., wood or cotton fibers), synthetic fibers (e.g., polyester or polypropylene fibers), or a combination of natural and synthetic fibers. Suitable backsheet materials may include breathable materials that permit vapors to escape from the diaper while still preventing exudates from passing through the backsheet.

A suitable absorbent core for use in the absorbent article 20 may comprise any absorbent material which is generally compressible, conformable, non-irritating to the wearer's skin, and capable of absorbing and retaining liquids such as urine and other certain body exudates. In addition, the configuration and construction of the absorbent core may also be varied (e.g., the absorbent core(s) or other absorbent structure(s) may have varying caliper zones, hydrophilic gradient(s), a superabsorbent gradient(s), or lower average density and lower average basis weight acquisition zones; or may comprise one or more layers or structures). In some embodiments, the absorbent core may comprise a fluid acquisition component, a fluid distribution component, and a fluid storage component.

Suitable absorbent cores of the present disclosure may comprise cellulosic airfelt material. For instance, such absorbent cores may comprise less than about 95%, 90%, 80%, 70%, 60, 40%, 50%, 40%, 30%, 20%, 10%, 5%, or even 1% of the cellulosic airfelt material as determined by weight. Additionally, such an absorbent core may be primarily comprised of an absorbent gelling material in amounts of at least about 5%, 10%, 20%, 30%, 40%, 50%, 60%, 70%, 80%, 85%, 90%, 95%, or even about 100% as determined by weight. Furthermore, a portion of the absorbent core may comprise a microfiber glue.

The outer cover layer 42 may be disposed on the outer surface 22 of the absorbent article and may cover the crotch panel 56 of the absorbent main body 38. The outer cover layer 42 may extend into and cover the front waist panel 52 and the back waist panel 54 of the main body 38. The outer cover layer may form a portion of the backsheet and/or the main body. The outer cover layer 42 may be directly joined to and cover a portion or all of the liquid impervious backsheet 60 of the main body 38. The central panel 80 of the front and back belt 84, 86 may be joined to the front waist panel 52 and the back waist panel 54 of the main body 38 through the outer cover layer 42. Thus, the outer cover layer 42 may be disposed between the front and back belt 84, 86 and the liquid impervious backsheet 60 of the main body 38. In one embodiment, the outer cover layer 42 may be coextensive with the liquid impervious backsheet 60.

The outer cover layer 42 may comprise a material separate from the material of the inner belt layer 83 and/or the outer belt layer 82 constituting the belt 40. The outer cover layer 42 may comprise two or more layers of materials. The outer cover layer 42 may comprise any known materials and may comprise materials used for the front and back belt 84, 86 as explained above. The outer cover layer 42 may comprise a single layer of nonwoven web of synthetic fibers. The outer cover layer 42 may comprise a single layer of hydrophobic, non-stretchable nonwoven material. The outer cover layer may comprise a film, a foam, a nonwoven, a woven material or the like and/or combinations thereof such as a laminate of a film and a nonwoven.

The front belt 84 and the back belt 86 are permanently or refastenably connected at the seams 32 to form a belt 40 having a ring-like configuration.

Front region 26 may be disposed on the front belt 84. Referring to FIGS. 3 and 4, the front region 26 comprises, in the longitudinal direction, an upper front region 212, a lower front region 216, an intermediate front region 214 between the upper front region 212 and the lower front region 216, and sides edges 260 opposed to each other. The front region 26 has a length L26 in a longitudinal direction, and the upper front region 212, the intermediate front region 214 and the lower front region 216 have lengths L212, L214, and L216, respectively in a longitudinal direction.

Still referring to FIGS. 3 and 4, the upper front region 212 extending in the transversal direction has side edges 262 opposed to each other, and comprise an area adjacent to each side edge 262. The area adjacent each side edge 262 may comprise an elastic material. The intermediate front region 214 extending in the transversal direction has side edges 264 opposed to each other, and comprises an area adjacent to each side edge 264. The area adjacent to each side edge 264 comprises an elastic material.

The combination of intermediate front region 214 with stretchability in both areas adjacent the side edges 264 is intended to provide good fit around the wearer's body in the waist as sides in the waist is relatively curved and front waist is relatively flat. Such design is especially advantageous for products for an adult. The lower front region 216 extending in the transversal direction and having side edges 266 opposed to each other has no stretchability especially in transversal direction so that substantially no or little pressure is given to the upper leg of a wearer.

The upper front region 212 and the intermediate front region 214 may have the same elastic material, elastic material arrangement, and/or elastic force. In one embodiment, the upper front region 212 and the intermediate front region 214 are not substantially different in elastic material and elastic material arrangement. Alternatively, the upper front region 212 may have different in at least one of elastic material and elastic material arrangement from the intermediate front region 214. In one embodiment, the upper front region 212 has a higher elastic force than the intermediate front region 214. Higher elastic force in the upper front region 212 than the intermediate front region 214 may provide desirable fit yet intend to make it more comfortable to wear given the body dimension corresponding to the intermediate front region is larger than the body dimension corresponding to the upper front region 212.

The front region 26 comprises, in the transversal direction, a first side region 261, a second side region 265 opposed to the first side region 261, and a central region 263 between the first side region 261 and the second side region 265. At least part of the central region 263 has no stretchability. In one embodiment, the central region 263 has no stretchability. With such a feature that at least part of the central region has no stretchability, the absorbent article may provide desirable fit and comfort as the body where the side regions are placed has a smaller curvature than the body where the central region is placed, and a more underwear-like look.

The upper front region 212 may comprise in the transversal direction an upper front first side region constituting the first side region 261, an opposite upper front second side region constituting the second side region 265, and an upper front central region constituting the central region 263, the upper front central region being between the upper front first side region and the upper front second side region. The intermediate front region 214 may comprise, in the transversal direction, an intermediate front first side region constituting the first side region 261, an opposite intermediate front second side region constituting the second side region 265, and an intermediate front central region constituting the central region 263, the intermediate front central region being between the intermediate front first side region and the intermediate front second side region. The intermediate front central region may have no stretchability.

In each of the upper front region and the intermediate front region, the side regions and the central region may differ in the elastic material or elastic material arrangement or elastic force. In one embodiment, in the upper front region, the elastic force in central region is lower than the elastic force in the side regions. In the embodiment, the central region in the upper front region may have no stretchability. In another embodiment, in the intermediate front region, the elastic force in central region is lower than the elastic force in the side regions. In the embodiment, the central region in the intermediate front region may have no stretchability. With such features that employment of elastic materials to provide an elastic force in the side regions while no stretchable material in the central region, the absorbent article can provide desirable fit and comfort as the body where the side regions are placed has a smaller curvature than the body where the central region is placed.

Elastic materials in the upper front region 212 may be disposed continuously along the entire front region in a transversal direction, or laterally interrupted by the main body 38.

Elastic materials in the intermediate front region 214 may be disposed continuously along the entire front region in a transversal direction, or laterally interrupted by the main body 38. In one embodiment, elastic materials located in the upper and intermediate front regions 212 and 214 are laterally interrupted by the main body 38.

When the upper front region 212 and/or the intermediate front region 214 overlap the main body 38, elastic materials in the front region may have one or more cuts in the area where the upper and/or intermediate front regions overlap the main body 38.

Front region 28 may be disposed on the back belt 86. The back region 28 comprises, in the transversal direction, an upper back region 222, a lower back region 226, an intermediate back region 224 between the upper back region 222 and the lower back region 226, and sides edges 280 opposed to each other. The front region 28 has a length L28 in a longitudinal direction, and the upper back region 222, the intermediate back region 224 and the lower back region 226 have lengths L222, L224, and L226, respectively in a longitudinal direction.

The back region 28 comprises, in the transversal direction, a first side region 281, a second side region 285 opposed to the first side region 281, and a central region 283 between the first side region 281 and the second side region 285. At least part of the central region 283 may have no stretchability.

The upper back region 222 may comprise in the transversal direction an upper back first side region constituting the first side region 281, an opposed upper back second side region constituting the second side region 285, and an upper back central region constituting the central region 283 between the upper back first side region and the upper back second side region. The intermediate back region 224 may comprise in the transversal direction an intermediate back first side region constituting the first side region 281, an opposed intermediate back second side region constituting the second side region 285, and an intermediate back central region constituting the central region 283 between the intermediate back first side region and the intermediate back second side region. The intermediate back central region may have no stretchability.

In each of the upper back region and the intermediate back region, the side regions and the central region may differ in the elastic material or elastic material arrangement or elastic force.

In one embodiment, in the upper back region, the elastic force in central region is lower than the elastic force in the side regions. In the embodiment, the central region in the upper back region may have no stretchability. In another embodiment, in the intermediate back region, the elastic force in central region is lower than the elastic force in the side regions. In the embodiment, the central region in the intermediate back region may have no stretchability. With such features that employment of elastic materials to provide an elastic force in the side regions while no stretchable area in the central region, the absorbent article can provide desirable fit and comfort as the body where the side regions are placed has a smaller curvature than the body where the central region is placed.

Elastic materials located in the upper back region 222 may be disposed continuously along the entire back region in a transversal direction, or may be laterally interrupted by the main body 38. Elastic materials located in the intermediate back region 224 may be disposed continuously along the entire back region in a transversal direction, or may be laterally interrupted by the main body 38. In one embodiment, elastic materials located in the upper and intermediate back regions 222 and 224 are laterally interrupted by the main body 38. When the upper back region 222 and/or the intermediate back region 224 overlap the main body 38, elastic materials in the back region may have one or more cuts in the area where the upper and/or intermediate back regions overlap the main body.

Still referring to FIGS. 3 and 4, the upper back region 222 extending in the transversal direction has side edges 282 opposed to each other, and comprise an area adjacent to each side edge 282. The area adjacent each side edge 282 may comprise an elastic material. The intermediate back region 224 extending in the transversal direction has side edges 284 opposed to each other, and comprises an area adjacent to each side edge 284. The area adjacent to each side edge 264 comprises an elastic material.

The lower back region 226 extending in the transversal direction has side edges 286 opposed to each other, and has no stretchability especially in transversal direction so that substantially no or little pressure is given to the upper leg of a wearer.

The upper back region 222 and the intermediate back region 224 may have the same elastic material, elastic material arrangement, and/or elastic force. In one embodiment, the upper back region 222 and the intermediate back region 224 are not substantially different in elastic material and elastic material arrangement. Alternatively, the upper back region 222 may have different in at least one of elastic material and elastic material arrangement from the intermediate back region 224. In one embodiment, the upper back region 222 has a higher elastic force than the intermediate back region 224. Higher elastic force in the upper back region 222 than the intermediate back region 224 may provide desirable fit yet intend to make it more comfortable to wear.

The elastic force of the upper front region 212 may be higher than the elastic force of the intermediate front region 214. Similarly, the elastic force of the upper back region 222 may be higher than the elastic force of the intermediate back region 224.

In the front region 26, the upper front region 212 and the intermediate front region 214 may be equally divided by longitudinal distance along the seam 32. Alternatively, the upper front region 212 may be shorter than the intermediate front region 214 by longitudinal distance along the seam 32. A longitudinal distance of the intermediate front region 214 may be about 100%-300% that of the upper front region 212. A longitudinal distance of the lower front region 216 may be no less than about 5%, 10%, or 15% of the longitudinal distance of the front region 26.

In the back region 28, the upper back region 222 and the intermediate back region 224 may be equally divided by longitudinal distance along the seam 32. Alternatively, the upper back region 222 may be shorter than the intermediate back region 224 by longitudinal distance along the seam 32. A longitudinal distance of the intermediate back region 214 may be about 100%-300% that of the upper back region 222. A longitudinal distance of the lower back region 226 may be no less than about 5%, 10%, or 15% of the longitudinal distance of the back region 28.

The crotch region 30 located between the front region 26 and the back region 28 in the longitudinal direction compirses a front crotch region 312, a back crotch region 316, and an intermediate crotch region 314 between the upper back region and the lower back region in the longitudinal direction. The front crotch region 312 and the back crotch region 316 are disposed distal (i.e., toward the transverse axis Ti) of each front and back regions 26 and 28. The front, intermediate and back crotch regions 312, 314, 316 extend in the transversal direction. The front crotch region 312 has no stretchability, and the back crotch region 316 may have no stretchability. Having no stretchable front crotch region 312, and back crotch region 316 optionally, the absorbent article of the present invention may allow wearers to adjust the height of product up/down on her body easily. In addition, it may reduce the potential uncomfortableness around the legs due to elastic force in these regions in conventional products. A longitudinal distance of the front crotch region 312 may be no less than about 5%, 10%, 15%, 20% or 50% of the longitudinal distance of the front region 26. A longitudinal distance of the back crotch region 316 may be no less than about 5%, 10%, 15%, 20% or 50% of the longitudinal distance of the back region 28.

The total longitudinal distance of the lower front region 216 and the front crotch region 312 may be about 100%-300% that of the upper front region 212. The total longitudinal distance of the lower back region 226 and the back crotch region 316 may be about 100%-300% that of the upper back region 222.

At least part of the crotch region 30 overlapping the absorbent core 62 has no stretchability, preferably along the transversal direction. With such a feature the absorbent article of the present invention may minimize the reduction of absorbent width in the presence of elasticity/stretchability; such a reduction may have potential to increase leakage. It also minimize the bunching of the absorbent width in the presence of elasticity/stretchability, resulting in a more comfortable wearing experience.

The elastic strands may be distributed amongst different regions in the front and/or the back regions. Elastic strands may be distributed evenly amongst the different regions in the front and/or the back regions. Elastic strands may also be distributed unevenly amongst different regions in the front and/or the back regions.

The elastic strands 300 may have a linear density between 200 to 2500. Linear density is the density of the elastic fibers in the elastic strand. The most commonly used unit for the linear density is the decitex, abbreviated dtex, which is the mass in grams per 10,000 meters (Dt). The linear density may be used to change the force profile. For example, one could reach a desired force profile by selecting the linear density of a single elastic strand, combining multiple elastic strands with a smaller linear density in close proximity to each other, and/or combining with other elastomeric materials.

The number of elastic strands in each zone may be changed according to the placement of the absorbent core. The force profile must be adjusted depending on the location and thickness of the absorbent core.

The gaps between the elastic strands 300 may be 2 mm, 3 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, or 22 mm, or 25 mm.

The absorbent article 20 may have a graphic disposed in the front region 26 and/or the back region 28. Graphics may be printed on the outermost nonwovens (e.g. the outer belt layer 82, or the outer cover layer 42), either on the garment facing side or the body facing side of these nonwovens. Graphics may also be printed on the garment facing side of the inner belt layer 83. Graphics of the present disclosure may be printed in part on the backsheet and in part on the belt layer.

Test Methods

These tests can be used for stretch laminates that comprise elastic strands, e.g. Spandex strands. Typically, the elastic strands are stretched and laminated between two nonwovens to make the stretch laminate. Since these tests will be done for each elastic region, it represents an average value for all the elastics in that region. These tests are suitable for pant type absorbent articles, comprising parallel strand elastics that run predominantly in the transverse direction of the product.

1. Elastic Force, F

Starting with a new product sample, i) mark the upper front region 212, intermediate front region 214, lower front region 216, upper back region 222, intermediate back region 224, and lower back region 226 in the front region 26 and/or back regions 28, ii) separate the belt 40 (or, front region 26 or back region 28) from the main body 38, and iii) cut off the upper, intermediate and lower regions.

Take the upper front region 212 and lay it down on the table in a relaxed state. Gently spread out the sample so it lies uniformly, while still being in a relaxed state. The stretch direction is referred to as the length direction and the direction perpendicular to the stretch direction is referred to the width direction for this test.

Draw 2 vertical lines that are parallel to the longitudinal center line, L1, and are at a distance of 50 mm on either side of the longitudinal center line, L1. These two lines should extend the full width of the region. If this region is not long enough to get this 100 mm long relaxed sample, a smaller length may be used. This smaller length should be ˜20 mm less than the minimum length of this region.

Measure the width of this upper front region, Ws-upper front.

Measure the tensile properties of this stretch laminate in a tensile tester as described below.
1) On an Instron or an MTS tensile tester, set the program to measure force as a function of strain.
2) Set the initial gage length to 50 mm. If it is not possible to get 50 mm gage length, use the maximum gage length possible.
3) Set the crosshead speed to 10 in./min.
4) Place the sample in between grips of the tensile tester, ensuring that the grips are wider than the sample.
5) Clamp the sample in the top grip and zero the load cell.
6) Clamp the sample in the bottom grip, while ensuring that there is some slack in the sample. The sample should not be under tension at this time.
7) Start the test. The tensile tester will collect load vs. displacement data. Record the distance between the grips when the force is 0.05 N. This is the adjusted gage length corresponding to zero strain. It is sometimes referred to as slack preload.
8) Record load vs. strain using the adjusted gage length as the starting length.
9) Continue collecting data until a strain of 100% is reached.
10) Report load in Newtons/cm at 50, 75 and 100% strains (F50-upper front, F75-upper front, and F100-upper front) using the width of the sample, Ws-upper front.

Repeat this measure for each of the remaining regions to be tested.

### EXAMPLES

### Example 1

A unibody type menstrual pant was produced according to descriptions in Table 1 below.

TABLE 1

| | | | | % |
|---|---|---|---|---|
| | | | | of seam |
| | | | | length |
| | Product width | 590 mm (Side to side) | | |
| | Product length | 585 | mm | |
| Front | Length of the front region | 120 | mm | |
| region | Lengths of 212 | 30 | mm | 25.0 |
| | 214 | 69 | mm | 57.5 |
| | 216 | 21 | mm | 17.5 |
| Back | Length of the back region | 120 | mm | |
| region | 222 | 30 | mm | 25.5 |
| | 224 | 72 | mm | 60.0 |
| | 226 | 18 | mm | 15.0 |
| Crotch | 312 | 8 | mm | 6.8 |
| region | 316 | 16 | mm | 13.3 |

| | | |
|---|---|---|
| Main body | Length of Topsheet | 585 mm (from front |
| | and outer cover | waist edge to back |
| | layer | waist edge) |
| Elastic | Waist elastics: number, | Front: 11, 280% |
| | pre-stretch % | Back: 11, 280% |
| | Hip elastics: number, | Front: 23, 180% |
| | pre-stretch % | Back: 24, 180% |
| | Outer cuff (Leg elastics: | 2, 200% |
| | number, pre-stretch % | |

Chinese consumers between ages 18-40 (n=12) were asked to wear an invention product at one night and a typical market product as a comparison product for another night during their period according to the usage habit as they normally use. The consumers were then asked to give comments on their experience for items indicated in Table 2 below.

TABLE 2

| | | Invention product | Comparison product* |
|---|---|---|---|
| Overall fit | | 8/12 | 4/12 |
| Wears like underwear | | 8/12 | 4/12 |
| Leg | Just right | 11/12 | 7/12 |
| tightness | Tight | 1/12 | 5/12 |
| Waist | Just right | 12/12 | 1/12 |
| tightness | Tight | 0/12 | 1/12 |

*Sofy size M (Unicharm, China)

8 out of 12 (67%) consumers responded that the invention product gave overall good fit while 4 out 12 consumers gave overall good fit for the comparison product.

As for a wearing comfort measurement, 8 out of 12 consumers commended the invention product feel like an underwear while 4 out 12 consumers commended for the comparison product.

11 out of 12 consumers felt right tightness around legs with only 1 out 12 felt tight while 5 out of 12 consumers responded tight feel around legs for the comparison product.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A disposable absorbent article having a longitudinal direction and a transversal direction perpendicular to the longitudinal direction, and comprising a front region, a back region, a crotch region between the front region and the back region in the longitudinal direction, and a main body comprising an absorbent core disposed to cover continuously the crotch region and at least part of the front region and back region, wherein the front and back regions are connected each other such that a first and a second seams are formed so that the absorbent article comprises leg openings and a waist opening,
wherein the front region comprises, in the longitudinal direction, an upper front region, a lower front region, and an intermediate front region between the upper front region and the lower front region, the front region comprising, in the transversal direction, a first side region, a second side region, and a central region between the first side region and the second side region,
wherein the back region comprises, in the longitudinal direction, an upper back region, a lower back region having side edges opposed to each other, and an intermediate back region between the upper back region and the lower back region, the back region comprising, in the transversal direction, a first side region, a second side region, and a central region between the first side region and the second side region,
wherein the crotch region comprise, in the longitudinal direction, a front crotch region connected to the front region, a back crotch region connected to the back region, and an intermediate crotch region between the upper back region and the lower back region,
wherein the intermediate front region comprises an area adjacent to each side edge, the side edge comprising an elastic material,
wherein the lower front region and the front crotch region have no stretchability, and
wherein at least part of the crotch region overlapping the absorbent core has no stretchability.

2. The disposable absorbent article of claim 1, wherein the lower back region and the back crotch region have no stretchability.

3. The disposable absorbent article of claim 1, wherein the intermediate front region has a lower elastic force than the upper front region.

4. The disposable absorbent article of claim 1, wherein the intermediate back region has a lower elastic force than the upper back region.

5. The disposable absorbent article of claim 1, wherein the central region in the front region has no stretchability.

6. The disposable absorbent article of claim 1, wherein the intermediate front region comprises, in the transversal direction, a first side region, a second side region opposed to the first side region, and a central region between the first side region and the second side region, wherein the central region has no stretchability.

7. The disposable absorbent article of claim 1, wherein the central region in the back region has no stretchability.

8. The disposable absorbent article of claim 1, wherein the intermediate back region comprises, in the transversal direction, a first side region, a second side region opposed to the first side region, and a central region between the first side region and the second side region, wherein the central region has no stretchability.

9. The disposable absorbent article of claim 1, wherein each of the front region and back region comprises at least one material continuous along the transversal direction.

10. The disposable absorbent article of claim 1, wherein a longitudinal distance of the intermediate front region is about 100%-300% that of the upper front region.

11. The disposable absorbent article of claim 1, wherein a total longitudinal distance of the lower front region and the front crotch region is about 100%-300% that of the upper front region.

12. The disposable absorbent article of claim 1, wherein the intermediate front region does not overlap the absorbent core.

13. The disposable absorbent article of claim 1, wherein core extends to the intermediate back region.

14. The disposable absorbent article of claim 1, wherein the crotch region comprises curved leg elastics.
